(19) United States
(12) Patent Application Publication
(10) Pub. No.: US 2024/0358733 A1
(43) Pub. Date: Oct. 31, 2024

(54) APPLICATION OF TYPHANEOSIDE IN PREPARATION OF MEDICINE FOR URIC ACID-LOWERING AND/OR KIDNEY INJURY

(71) Applicant: Institute of Chinese Materia Medica, China Academy of Chinese Medical Sciences, Beijing (CN)

(72) Inventors: Aihua Liang, Beijing (CN); Jing Meng, Beijing (CN); Jingzhuo Tian, Beijing (CN)

(21) Appl. No.: 18/646,814

(22) Filed: Apr. 26, 2024

(30) Foreign Application Priority Data
Apr. 27, 2023 (CN) 202310465754.3

(51) Int. Cl. A61K 31/7048; A61P 7/00; A61P 13/12

(52) U.S. Cl. CPC A61K 31/7048; A61P 7/00; A61P 13/12

(57) ABSTRACT

The present disclosure herein belongs to the field of biomedicine, and discloses an application of typhaneoside in preparation of a medicine for uric acid-lowering and/or hyperuricemic kidney injury, wherein the typhaneoside is the sole active ingredient in the preparation of a medicine for uric acid-lowering and/or hyperuricemic kidney injury. The typhaneoside being able to significantly reduce the blood uric acid level, at the same time, the typhaneoside is able to improve hyperuricemic kidney injury by lowering the uric acid deposition in the kidneys. As a drug for anti-hyperuricemia and improving hyperuricemic kidney injury, typhaneoside shows significant application prospects and is widely used in the market.

### CROSS-REFERENCE TO RELATED APPLICATION

The present application claims priority from Chinese Patent Application No. 202310465754.3 filed on Apr. 27, 2023, the contents of which are incorporated herein by reference in their entirety.

### TECHNICAL FIELD

The present disclosure herein relates to the field of biomedicine, and particularly relates to an application of typhaneoside in preparation of a medicine for uric acid-lowering and/or hyperuricemic kidney injury thereof.

### BACKGROUND

Hyperuricemia is a metabolic syndrome in which abnormal purine metabolism leads to an increase in uric acid production and/or a decrease in uric acid excretion resulting in abnormally high blood uric acid levels. When the concentration of uric acid in the body is too high, urate crystals can be precipitated and deposited in the kidneys, joints and other tissues, leading to uric acid nephropathy or gout. Hyperuricemia is also an independent risk factor for chronic kidney disease, diabetes mellitus, hypertension and metabolic syndrome and other diseases, which can increase the risk of death. Therefore, there is a significant clinical need to treat hyperuricemia. Currently, the most commonly used drugs in the clinic are allopurinol and febuxostat, which are xanthine oxidase inhibitors that inhibit uric acid synthesis, and probenecid and benzbromarone, which promote uric acid excretion. Despite the efficacy of these drugs, there are different degrees of adverse reactions. Allopurinol can cause severe or even fatal skin damage in some patients; febuxostat has unpredictable cardiovascular risks and can increase the risk of death; and benzbromarone can cause severe hepatotoxicity. In addition, hyperuricemia can lead to kidney injury, but the clinical application of the current drug instead of kidney protective effect, some will exacerbate the degree of kidney injury, such as allopurinol. For these reasons, there is a high demand for the development of drugs with renoprotective effects for treating hyperuricemia clinically.

Typhaneoside, a flavonoid compound, has good prospects for medicinal use as the compound, and has pharmacological effects such as improving microcirculation, reducing oxygen consumption of heart and brain tissues, promoting coagulation, vasodilating, anti-inflammatory, lowering blood lipids, and anti-atherosclerosis. Chinese patent CN113712985A discloses an application of typhaneoside in preparation of a medicine for treating inflammatory bowel diseases. The disclosure uses typhaneoside to treat the DSS-induced colitis model in C57BL/6J mice, and inflammation-related parameters such as body weight loss, disease activity index, and splenic weight of the mice are improved, and histological staining reveals that intestinal inflammation in the mice is suppressed, and the pathology scores are decreased, the specific mechanism of the anti-inflammatory effect was further investigated, and it was found that typhaneoside could inhibit the activation of NF-κB pathway in mouse intestinal epithelial cells and reduce the expression of inflammatory factors in mouse intestinal epithelial cells, and the monomer of this traditional Chinese medicine, typhaneoside, could provide a new way for the preparation of therapeutic drugs for IBD and its related diseases in the future. Chinese patent CN114939123A discloses a therapeutic preparation for promoting wound repair, the therapeutic preparation comprising typhaneoside and a pharmaceutically acceptable carrier. The disclosure was found to be able to effectively promote the protein expression of the cell migration-promoting protein N-cadherin in human skin fibroblasts, and to effectively inhibit the protein expression of the cell migration inhibitory protein E-cadherin in human skin fibroblasts, thereby effectively promoting the migration of the human skin fibroblasts; moreover, the disclosure was found that the typhaneoside was able to effectively promote the expression of Collagen I and Collagen III proteins in human skin fibroblasts, thereby promoting the secretion of Collagen I and Collagen III proteins by human skin fibroblasts to promote wound repair.

However, no relevant experimental studies and application of typhaneoside in preparation of a medicine for uric acid-lowering and/or hyperuricemic kidney injury have been found in the current state of the art.

### SUMMARY

In order to solve the above technical problems, the present disclosure provides a new pharmaceutical use of typhaneoside, specifically providing an application of typhaneoside in preparation of a medicine for uric acid-lowering and/or hyperuricemic kidney injury, which further comprises a medically acceptable carrier.

In order to achieve the above purpose, the present disclosure adopts the following technical solutions:

The present disclosure provides an application of typhaneoside in preparation of a medicine for uric acid-lowering and/or hyperuricemic kidney injury.

Preferably, the molecular formula of the typhaneoside is C₃₄H₄₂O₂₀, and the chemical structure formula is shown in Formula I:

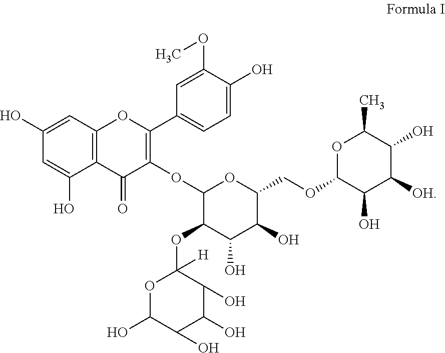 [SMILES: COc1cc(-c2oc3cc(O)cc(O)c3c(=O)c2OC2O[C@H](CO[C@@H]3O[C@@H](C)[C@H](O)[C@@H](O)[C@H]3O)[C@@H](O)[C@H](O)[C@H]2OC2OC(O)C(O)C(O)C2O)ccc1O]

Preferably, the drug comprises typhaneoside and a medically acceptable carrier, wherein the typhaneoside is the sole active ingredient.

Preferably, the carrier comprises at least one of a diluent, a binder, an absorbent, a disintegrant, a dispersant, a wetting agent, a co-solvent, a buffer and a surfactant.

Preferably, the uric acid-lowering drug comprises a drug for treating hyperuricemia.

Preferably, the dosage form of the drug is solid, liquid or gas.

Further preferably, the dosage form of the solid is powder, tablet, granule, pill, hard capsule, soft capsule, cream, ointment, hard paste, gel, paste, dispersion or patch; the dosage form of the liquid is solution, suspension, injection, syrup, liniment, lotion, tincture or elixir; and the dosage form of the gas is aerosol or spray.

Preferably, the drug is administered by at least one of oral, sublingual, oral mucosal, intravenous, intramuscular, intraperitoneal, subcutaneous, transdermal, nasal, rectal routes.

The present disclosure further provides a uric acid-lowering drug comprising the above-described typhaneoside and a medically acceptable carrier, wherein the typhaneoside is the sole active ingredient.

Preferably, the carrier comprises at least one of a diluent, a binder, an absorbent, a disintegrant, a dispersant, a wetting agent, a co-solvent, a buffer and a surfactant.

The present disclosure further provides a drug for treating hyperuricemic kidney injury comprising the above-described typhaneoside and a medically acceptable carrier, wherein the typhaneoside is the sole active ingredient.

Preferably, the carrier comprises at least one of a diluent, a binder, an absorbent, a disintegrant, a dispersant, a wetting agent, a co-solvent, a buffer and a surfactant.

The beneficial effects of the present disclosure are:
the present disclosure provides a new application of typhaneoside, and specifically provides the application of typhaneoside in preparation of a medicine for uric acid-lowering and/or hyperuricemic kidney injury, the typhaneoside being able to significantly reduce the blood uric acid level, showing significant uric acid-lowering effect; at the same time, the typhaneoside is able to improve hyperuricemic kidney injury by lowering the uric acid deposition in the kidneys. As a drug for anti-hyperuricemia and improving hyperuricemic kidney injury, typhaneoside shows significant application prospects and is widely used in the market.

### BRIEF DESCRIPTION OF THE DRAWINGS

FIG. 1 shows the hexamine silver staining graph of the effect of the administration of typhaneoside on urate deposition in rat kidney tissues, wherein, A is the control group; B is the model group; C is the typhaneoside low dose group; D is the typhaneoside high dose group; E is the isorhamnetin low dose group; and F is the isorhamnetin high dose group.

FIG. 2 shows the pathological observation graph of rat kidney tissues, wherein, A is the control group; B is the model group; C is the typhaneoside low dose group; D is the typhaneoside high dose group; E is the isorhamnetin low dose group; and F is the isorhamnetin high dose group.

### DETAILED DESCRIPTION OF THE EMBODIMENTS

The description of the following examples is only intended to assist in understanding the method of the present disclosure and its core ideas. It should be noted that for a person of ordinary skill in the art, several improvements and modifications can be made to the present disclosure without departing from the principles of the present disclosure, which also fall within the scope of protection of the claims of the present disclosure. The following description of the disclosed examples enables a person skilled in the art to realize or use the present disclosure. Various modifications to these examples will be apparent to those skilled in the art, and the general principles defined herein may be realized in other examples without departing from the spirit or scope of the present disclosure. Accordingly, the present disclosure will not be limited to those examples shown herein, but may be applied to a broader scope consistent with the principles and novel features disclosed herein. While any methods and materials similar or equivalent to those described herein may be used in the implementation or testing of the present disclosure, preferred methods and materials are enumerated herein.

Unless otherwise defined, all technical and scientific terms used herein have the same meaning as is commonly understood by those of ordinary skill in the art to which the present disclosure belongs.

### EXAMPLE 1 EFFECT OF TYPHANEOSIDE ON BLOOD URIC ACID LEVEL IN RATS WITH HYPERURICEMIA

### 1. Materials and Methods

### (1) Grouping of Animals and Mode of Administration

There were 48 Male SPF grade SD rats, weighing 200-220 g, purchased from Beijing Vital River Laboratory Animal Technology Co., Ltd., license No. SCXK (Beijing) 2016-0006. The animals were housed in a barrier environment animal laboratory, with automatic temperature control (temperature 20-26° C.), relative humidity 40-70%, and full fresh air. Artificial light was adopted with a 12-hour light-dark cycle. Group experiments were started after 1 day of acclimatization. Animals were fed with standard rat pellet feed, provided by Beijing Keao Xieli Feed Co. Ltd., with purified water.

The rats were randomly divided into 6 groups of 8 rats each, which were control group, model group, typhaneoside low dose group (25 mg/kg) and typhaneoside high dose group (50 mg/kg), isorhamnetin low dose group (25 mg/kg) and isorhamnetin high dose group (50 mg/kg).

### (2) Establishment of Adenine Combined with Ethambutol-Induced Hyperuricemia Model in Rats

Adenine was purchased from Sigma-Aldrich (USA), and 0.5% CMC-Na was used to formulate adenine into a suspension at a concentration of 20 mg/mL, ready-to-use, with an administration volume of 5 mL/kg and a dosage of 100 mg/kg. Ethambutol hydrochloride tablets were purchased from Chengdu Jinhua Pharmaceutical Co., Ltd, and 0.5% CMC-Na was used to formulate ethambutol hydrochloride tablets into a suspension at a concentration of 50 mg/ml, ready-to-use, with an administration volume of 5 mL/kg and a dosage of 250 mg/kg.

The modeling drug adenine 100 mg/kg and ethambutol hydrochloride 250 mg/kg were administered by gavage in the model group, typhaneoside high and low dose groups, and isorhamnetin high and low dose groups, respectively, from day 1, and both were administered once a day for a total of 7 days, to prepare a hyperuricemia rat model. An equal amount of 0.5% CMC-Na was administered by gavage in the control group at the relative time.

After modeling, the low dose and high dose groups of typhaneoside and isorhamnetin were administered by gavage at a dose of 25 mg/kg and 50 mg/kg in the low dose and high dose groups, respectively, and an equal volume of 0.5% CMC-Na was administered by gavage in the control group and the model group. the drugs were administered once a day at 1 h after modeling for 7 days.

### (3) Sample Processing and Statistical Methods

Observation and detection indicators: fasting for 16 hours after the last administration, blood was collected from the abdominal aorta of rats in each group, centrifuged at 3500 rpm for 15 minutes after standing, separated and collected serum, and the content of uric acid in the serum was measured using an Automatic Chemical Analysis.

Statistical methods: data were expressed as mean±standard deviation (x̅±S) and analyzed by one-way ANOVA. Each administration group was compared with the model group separately.

### 2. Results

Results Table 1 showed that the serum uric acid level in the model group was significantly higher (p<0.05) compared to the control group, indicating successful modeling. Compared with the model group, the administration of different doses of typhaneoside had a significant reducing effect on the blood uric acid level of the animals with hyperuricemia, while the administration of different doses of isorhamnetin did not show a significant reducing effect on the blood uric acid level of the animals with hyperuricemia. The results suggested that the active ingredient in the present disclosure, typhaneoside, had a significant effect of reducing blood uric acid.

TABLE 1
Effects of different doses of typhaneoside and isorhamnetin
on blood uric acid levels in rats with hyperuricemia

| | Dosages | Blood uric acid |
|---|---|---|
| Groups | (mg/kg) | (μmol/L) |
| Control group | — | 111.70 ± 12.92 |
| Model group | — | 144.43 ± 31.20* |
| Typhaneoside | 25 | 94.78 ± 21.14^{##} |
| low dose group | | |
| Typhaneoside | 50 | 78.16 ± 31.28^{##} |
| high dose group | | |
| Isorhamnetin | 25 | 118.56 ± 26.76 |
| low dose group | | |
| Isorhamnetin | 50 | 105.74 ± 42.62 |
| high dose group | | |

Note:
compared with the control group,
*indicates P < 0.05; compared with the model group,
^{##}indicates P < 0.01.

### EXAMPLE 2 IMPROVEMENT EFFECT OF TYPHANEOSIDE ON KIDNEY INJURY IN RATS WITH HYPERURICEMIA

### 1. Materials and Methods

The establishment of the model as well as the experimental grouping and administration were the same as in Example 1.

(1) Kidney function biochemical indicators detection: the method of blood sampling and serum collection was the same as in Example 1, and an Automatic Chemical Analysis was used to detect the content of serum creatinine (SCr) by using the creatine oxidase method, and to detect the content of blood urea nitrogen (BUN) by using the urease method and the kinetic method.

(2) Pathological examination of kidney tissue: anesthetize rats, dissect, and remove kidney tissue. The kidney tissues were fixed with 10% neutral formalin. After sufficient fixation, sampling, gradient dehydration, xylene transparency, paraffin embedding, and preparation of 3 μm paraffin sections were performed according to routine procedures. The kidney tissue lesions as well as urate deposition in the kidney tissues were examined by light microscopy using HE (hematoxylin & eosin) staining and hexamine silver staining, respectively.

### 2. Results

### (1) Effect of Typhaneoside on Urate Deposition in Rat Kidney Tissues

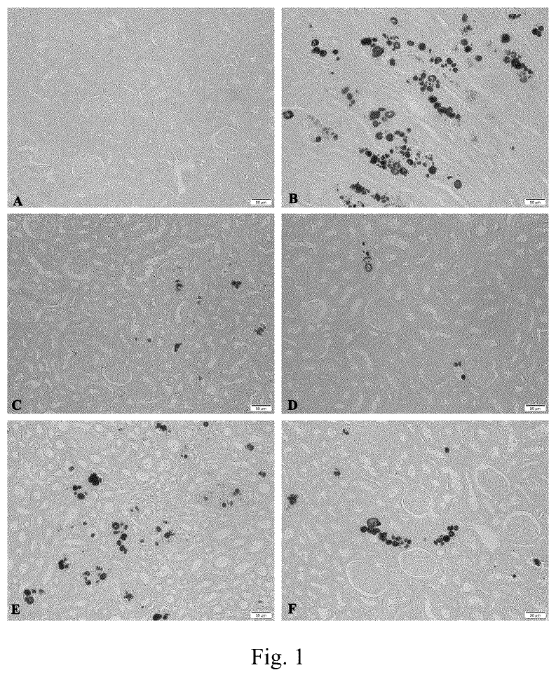

As can be seen from FIG. 1, urate crystals deposition was not observed in the control group, and a large number of urate crystals deposition was observed in the kidney tissue in the model group. The deposited urate crystals were significantly reduced after the administration of typhaneoside. In addition, the amount of deposited urate crystals after the administration of isorhamnetin was only slightly reduced compared with that in the model group. The results suggested that typhaneoside can significantly reduce the urate crystals deposition in the kidney tissues of rats with hyperuricemia.

### (2) Effects of Typhaneoside on Biochemical Indicators Related to Kidney Injury in Rats

The results in Table 2 indicated that both serum BUN and SCr levels were significantly higher in the model group compared to the control group (p<0.001), suggesting that the rats with hyperuricemia exhibited some kidney injury and the modeling was successful. The serum BUN level was significantly reduced after the administration of typhaneoside compared with that of the model group (p<0.01), whereas the serum BUN and SCr levels did not show significant reduction after the administration of isorhamnetin compared with that of the model group. It is suggested that typhaneoside had an improvement effect on kidney injury caused by hyperuricemia.

TABLE 2
Effects of different doses of typhaneoside and isorhamnetin on blood
BUN and SCr levels in rats with hyperuricemic kidney injury

| | Dosages | BUN | SCr |
|---|---|---|---|
| Groups | (mg/kg) | (mmol/L) | (μmol/L) |
| Control group | — | 4.34 ± 0.52 | 31.46 ± 3.82 |
| Model group | — | 11.87 ± 2.27*** | 49.09 ± 9.42*** |
| Typhaneoside | 25 | 6.73 ± 1.67^{##} | 43.71 ± 5.23 |
| low dose group | | | |
| Typhaneoside | 50 | 7.67 ± 1.00^{##} | 45.30 ± 5.65 |
| high dose group | | | |
| Isorhamnetin | 25 | 7.90 ± 2.48 | 41.66 ± 5.72 |
| low dose group | | | |
| Isorhamnetin | 50 | 8.26 ± 3.99 | 47.12 ± 12.00 |
| high dose group | | | |

Note:
compared with the control group,
***indicates P < 0.001; compared with the model group,
^{##}indicates P < 0.01.

### (3) Effects of Typhaneoside on the Pathology of Rat Kidney Tissues

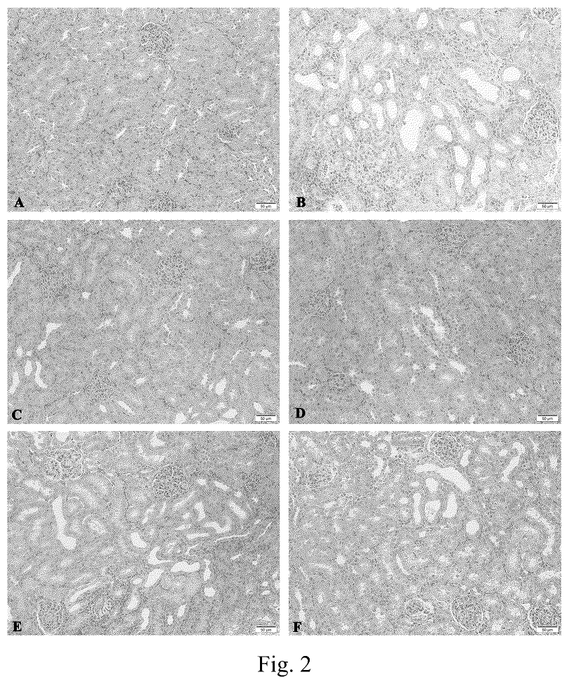

As can be seen from FIG. 2, in the control group, the tissue structures of renal interstitium, renal tubules and glomerular were basically normal, the cells were neatly arranged, and no pathological changes such as tubular dilatation, degeneration or necrosis were observed. In the model group, the glomerular morphology was basically normal in the kidney tissues, but pathological changes such as tubular wall thinning, severe dilatation, epithelial cell detachment and eosinophilic degeneration were observed, and obvious inflammatory cell infiltration was also observed. In the typhaneoside low dose group, the glomerular morphology was basically normal in the kidney tissues, and the phenomenon of tubular dilatation was reduced compared with that of the model group, but pathological changes such as tubular dilatation and tubular wall thinning, inflammatory cell infiltration and tubular epithelial cell detachment could be observed locally. In the typhaneoside high dose group, the glomerular morphology was basically normal in the kidney tissues, the tubular dilatation phenomenon was significantly reduced, and only occasional slight tubular dilatation could be observed locally, and pathological changes such as slight tubular epithelial cell detachment and inflammatory cell infiltration could be observed. In the isorhamnetin high and low doses groups, pathological changes such as severe renal tubular dilatation and epithelial cell detachment were still observed, especially in the low dose group, obvious inflammatory cell infiltration and eosinophilic degeneration were observed.

The combination of hexamine silver staining results, biochemical results and pathological results suggested that the active ingredient in the present disclosure, typhaneoside, may have a significant protective effect on kidney injury combined with hyperuricemia by decreasing uric acid deposition in the kidneys.

The above is a further description of the present disclosure in conjunction with specific examples, but these examples are merely exemplary and do not constitute any limitation on the scope of the present disclosure. It should be understood by those skilled in the art that the details and forms of the technical solution of the present disclosure may be modified or replaced without deviating from the spirit and scope of the present disclosure, but these modifications and replacements fall within the scope of protection of the present disclosure.

What is claimed is:

1. Application of typhaneoside in preparation of a medicine for uric acid-lowering and/or hyperuricemic kidney injury.

2. The application according to claim 1, wherein the molecular formula of the typhaneoside is C₃₄H₄₂O₂₀, and the chemical structure formula is shown in formula I:

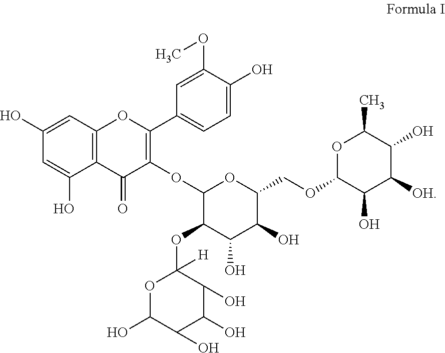 [SMILES: COc1cc(-c2oc3cc(O)cc(O)c3c(=O)c2OC2O[C@H](CO[C@@H]3O[C@@H](C)[C@H](O)[C@@H](O)[C@H]3O)[C@@H](O)[C@H](O)[C@H]2OC2OC(O)C(O)C(O)C2O)ccc1O]

3. The application according to claim 1, wherein the drug comprises typhaneoside and a medically acceptable carrier, wherein the typhaneoside is the sole active ingredient.

4. The application according to claim 3, wherein the carrier comprises at least one of a diluent, a binder, an absorbent, a disintegrant, a dispersant, a wetting agent, a co-solvent, a buffer and a surfactant.

5. The application according to claim 1, wherein the uric acid-lowering drug comprises a drug for treating hyperuricemia.

6. The application according to claim 1, wherein the dosage form of the drug is solid, liquid or gas.

7. A uric acid-lowering drug, wherein comprising the typhaneoside according to claim 1 and a medically acceptable carrier, wherein the typhaneoside is the sole active ingredient.

8. The uric acid-lowering drug according to claim 7, wherein the carrier comprises at least one of a diluent, a binder, an absorbent, a disintegrant, a dispersant, a wetting agent, a co-solvent, a buffer and a surfactant.

9. A drug for treating hyperuricemic kidney injury, wherein comprising the typhaneoside according to claim 1 and a medically acceptable carrier, wherein the typhaneoside is the sole active ingredient.

10. The drug according to claim 9, wherein the carrier comprises at least one of a diluent, a binder, an absorbent, a disintegrant, a dispersant, a wetting agent, a co-solvent, a buffer and a surfactant.
